(19) United States
(12) Patent Application Publication
(10) Pub. No.: US 2024/0301160 A1
(43) Pub. Date: Sep. 12, 2024

(54) ENZYMATIC RECYCLING OF RECYCLED POLYETHYLENE TEREPHTHALATE BY CUTINASES

(71) Applicant: SOCIETE DES PRODUITS N ÉSTLÉ S.A., Vevey (CH)

(72) Inventors: TIM BORNER, Epalinges (CH); NINA CHRISTINA MARIA ROOTHANS, TX Delft (NL)

(21) Appl. No.: 18/259,166

(22) PCT Filed: Dec. 13, 2021
(86) PCT No.: PCT/EP2021/085364

§ 371 Date: Jun. 23, 2023

(30) Foreign Application Priority Data
Dec. 24, 2020 (EP) 20217177.3

(51) Int. Cl. C08J 11/10; C12P 7/62

(52) U.S. Cl. CPC C08J 11/105; C12P 7/62; C08J 2367/02

(57) ABSTRACT

The present invention relates generally to the field of degrading recycled polyethylene terephthalate (rPET), for example rPET layers in multilayer packaging. For example, the present invention relates to a method of degrading rPET comprising the step of subjecting the rPET to at least one cutinase. The rPET may be a rPET-based layer in a multilayer packaging structure comprised in a packaging. Remarkably, the subject matter of the present invention allows the selective degradation of rPET containing layers in multilayer packaging materials.

### FIELD OF THE INVENTION

The present invention relates generally to the field of degrading recycled polyethylene terephthalate (rPET), for example rPET layers in multi-layer packaging. For example, the present invention relates to a method of degrading rPET comprising the step of subjecting the rPET to at least one cutinase. The rPET may be a rPET-based layer in a multilayer packaging structure comprised in a packaging. Remarkably, the subject matter of the present invention allows the selective degradation of rPET containing layers in multi-layer packaging materials.

### BACKGROUND OF THE INVENTION

Plastic production has been increasing for over the last six decades, reaching 348 million tonnes in 2017 (Plastics Europe, 2018). Packaging is the major sector of plastic usage, with almost 40% of the market demand (Plastics Europe, 2018). It consists for a large part of single-use plastics, which have a short lifetime, turning to waste shortly after being acquired by the consumer. It is common knowledge that plastic accumulation is a current major environmental concern, resulting from the high resistance of plastics to degradation, together with improper disposal or deposition of waste in landfills. Yet, efforts have been made over the past years to avoid plastic deposition in landfills (Plastics Europe, 2018). Nevertheless, a large amount of packaging plastics still ends up as waste, so efficient recycling technologies are needed to simultaneously minimize the amount of produced waste and the resource consumption to produce plastics.

Polymers used in packaging can be divided into two main groups: the ones with a carbon-carbon backbone [e.g., polypropylene (PP), polyethylene (PE), polyvinyl chloride (PVC) and polystyrene (PS)] and those with a heteroatomic backbone [e.g., polyesters and polyurethanes (PU)]. The high energy required to break C—C bonds makes hydrocarbons very resistant to degradation (Microb Biotechnol, 10(6), 1308-1322). On the other hand, polyesters and polyurethanes have hydrolysable polyester bonds so they are less resilient to abiotic and biotic degradation.

The most common polyester is polyethylene terephthalate (PET) (Plastics Europe, 2018). Because of its abundant usage, the ability to recycle PET is a key focus of the industry. According to a recent Plastics Recyclers Europe evaluation, the PET recycling capacity for Europe was 2.1 million tonnes in 2017. In order to reduce the usage of virgin PET the use of recycled PET (rPET) is increasing in the industry, both, as the only PET source in a packaging material, as well as in combination with virgin PET to result in a composite virgin PET and rPET material. Recently, for example bottles made only out of rPET have been marketed. As a consequence, according to a IHS Markit analysis for example in the US rPET makes up about 12 to 14 percent of the PET packaging resin produced and consumed annually.

Plastic packaging is usually not composed of one single polymer. Instead, blends or multiple layers of different polymers are often required to obtain certain properties (elasticity, hydrophilicity, durability or water and gas barrier) related to the specific application of the plastic (Process Biochemistry, 59, 58-64). Also, packaging materials generally contain adhesives, coatings and additives, such as plasticizers, stabilizers and colorants (Philos Trans R Soc Lond B Biol Sci, 364(1526), 2115-2126). This makes the recycling of some packaging materials very difficult.

Current plastic waste recycling technologies predominantly consist of thermo-mechanical processes, while chemical recycling is in its early industrialization phase. Mechanical recycling requires clean input waste streams that may be achieved through prior cleaning and separation steps in the case of contaminated and complex packaging structures, respectively. Thus, the recycling rates of multilayer packaging today are very low. Instead, multilayer packaging is mostly incinerated or ends up in landfills. Besides, the mechanical recycling process often results in downgraded plastics with decreased properties and limited food grade quality, thus losing their original value and application. These materials are then typically used for lower-value secondary products. On the other hand, chemical recycling processes are being developed to enable the recovery of the polymer's building blocks that can be used to remake the plastic. However, this process is economical and energetically costly and usually requires extreme conditions and harsh chemicals. These technologies are thus not ideal for complex, multilayer plastic materials (Process Biochemistry (2017), 59, 58-64).

A technology enabling the selective removal and recycling of each component of multilayer plastic packaging would provide the possibility of reproducing the original packaging and expanding recycling to mixed plastic packaging waste and materials.

Enzymes are very selective towards their substrate, so they offer a high potential to be applied in recycling processes. Enzymes would enable the selective decomposition of each layer into either the starting building blocks, which can be used for subsequent production of new plastics or as added-value chemicals. The enzymatic and microbial degradation of recalcitrant plastics has been increasingly studied over the past years, with particular focus on PET (Microb Biotechnol, 10(6), 1302-1307). Even though the enzymatic degradation of plastic is difficult, there are enzymes capable of degrading polyesters used in the production of plastic packaging. The degradation efficiency of enzymes however varies with different classes and types of enzymes, and the conditions under which the experiments were carried out highly influence the extent of degradation. In addition, the polymer properties, e. g., crystallinity and composition, also have a strong influence on the rate of degradation.

Even though efforts have been made to increase the efficiency of enzymatic degradation of polymers, most studies were performed on pure materials. Although these studies provide a good initial insight on the enzymatic degradation of plastics, they are not representative of actual packaging materials as polymers are not isolated in this case and additives may be present. Moreover, a deep understanding of the effect of experimental conditions, enzyme properties and polymer properties on the degradation process is lacking.

Therefore, to design a selective recycling process for multi-layer packaging is of high importance.

It would therefore be desirable to have available a process that can be used to selectively degrade (delaminate) rPET-based layers in multi-layer packaging that is cost efficient, results in high quality materials and does not require harsh processing conditions.

While it is known that PET can be degraded by cutinases, Nature Scientific Reports (2019) 9:16038, the prior-art lacks, to the best of the inventor's knowledge, information on enzymatic hydrolysis of recycled PET (rPET). Typically, to compensate for the reduced quality of rPET compared to virgin PET that is caused by the reduction in molecular weight through thermal hydrolysis during mechanical recycling, chain extenders are commonly used (D. S. Achilias (Ed.), Mater. Recycl. Trends Perspect., InTech, Rijeka, Croatia (2012), pp. 85-114). Such chemical modifications increase the molecular weight, lead to partial cross-linking and alter the overall chemistry of rPET compared to PET (see Torres et al. 2001, 79(10), 1816-1824). Hence, it is known that the composition and properties of rPET and PET are different. For example, Packag Technol Sci. 2020; 33:359-371, lists some of these differences. As one result, for example, rPET has typically higher crystallinity than PET (Thermochimica Acta Volume 683, January 2020, 178472). A higher degree of crystallinity can have a negative impact on the hydrolysis efficiency of the enzymes. Moreover, chemical modifications through extenders, for example, will impact enzymatic hydrolysis. Hence, it cannot be concluded that—because cutinases are known to be able to degrade PET—that they can also be used to degrade rPET.

It would however be desirable to have available a process to enzymatically hydrolyze rPET, for example rPET food packaging, such as rPET bottles, and delaminate one or more rPET layers present in a multilayer packaging, which would allow to produce the monomers for re-producing virgin recycled PET (rPET), which would in turn allow to reuse recycled rPET for food packaging application or other high-value applications, for example.

Importantly, it is known that the crystallinity of pure PET can be changed through melting and cooling (quenching), e.g., via extrusion, but this approach is not possible for multilayer structures due to the different polymer properties that cannot be separated and thus make, for example, an extrusion process inapplicable. Hence, an efficient enzymatic depolymerization and delamination process for rPET/PET containing multilayer packaging is highly needed.

Any reference to prior art documents in this specification is not to be considered an admission that such prior art is widely known or forms part of the common general knowledge in the field.

### SUMMARY OF THE INVENTION

The objective of the present invention was, hence, to enrich or improve the state of the art and in particular to provide the art with a method to degrade rPET, for example applied to a rPET layer in a multi-layer packaging that does not require prior separation of layers, does not require harsh chemicals and/or harsh conditions, and offers economic and environmental advantages, or to at least provide a useful alternative to solutions available in the art.

The inventors were surprised to see that the objective of the present invention could be achieved by the subject matter of the independent claim. The dependent claims further develop the idea of the present invention.

Accordingly, the present invention provides a method of delaminating (and depolymerizing) rPET comprising the step of subjecting the rPET to at least one cutinase.

As used in this specification, the words “comprises”, “comprising”, and similar words, are not to be interpreted in an exclusive or exhaustive sense. In other words, they are intended to mean “including, but not limited to”.

The present inventors have shown that cutinases can efficiently be used to degrade rPET. The inventors have obtained particular promising results with the cutinases Thf_Cut, Thc_Cut1, Thc_Cut2, and cutinase-like enzyme BC-CUT-013. Remarkably, all cutinase enzymes could be efficiently used to degrade rPET, also in rPET/PET composite materials. They could also be used to selectively degrade rPET-containing layers in multilayer packaging. For example in the case of PE based multilayer packaging structure that comprises an rPET-based layer, it will be possible by using cutinases to selectively degrade the rPET-based layer, so that the rPET monomers can be recovered, and the PE-based backbone of the multilayer packaging structure can be liberated and subjected to PE recycling. The clean state of the resulting PE allows it that the recycled PE can be recycled for high-value applications.

### BRIEF DESCRIPTION OF THE DRAWINGS

Additional features and advantages of the present invention are described in, and will be apparent from, the description of the presently preferred embodiments which are set out below with reference to the drawings in which:

FIG. 1 shows the increase of total hydrolysis products (mM) released from post-consumer PET with 30% recycled PET content using the four different cutinases and cutinase-like enzymes, respectively: Thf_Cut (⬆, diamond), Thc_Cut2 (Δ, triangle), Thc_Cut1 (∘, circle) and BC-CUT-013 (□, square). The experiment also included a negative control consisting (•, filled circle) of 0.1M PBS at pH 7. The reactions were carried out for 7 days at 37° C. and pH 7 with 20-25 mg rPET substrate grounded to 0.2-0.5 mm. Each symbol represents the average of two reactions. Product concentration (TPA, BHET, MHET) were determined by HPLC. The enzyme loading was typically adjusted to 6 μg protein/mg polymer.

FIGS. 2A and 2B show the individual amount of each hydrolysis products released by four different enzymes after 2 days (A) and 7 days (B) reaction time using post-consumer PET with 30% recycled PET content. The reactions were carried out at 37° C. and pH 7, with 20-25 mg rPET substrate grounded to 0.2-0.5 mm for 2 days (A) and 7 days (B), respectively. The differently colored fractions in the product bars indicate the concentration of the hydrolysis products TPA (white), MHET (grey) and BHET (black) of the reaction mixture determined by HPLC. Each bar represents the average concentration of total products from duplicate reactions with their respective maximum and minimum value. The typically used enzyme loading was set to 6 μg protein/mg polymer per reaction.

FIG. 3 shows the total product concentration (BHET+MHET+TPA) after 7 days of enzymatic hydrolysis of post-consumer PET with 75% recycled content at pH 7.5 (black), 8 (grey), and 8.2 (white). A negative control was performed with rPET solely in 0.1 M PBS buffer. The reactions were carried out in 4 ml glass vials at 37° C. with 20-25 mg rPET substrate grounded to 0.2-0.5 mm. Each bar represents the average concentration of total products from duplicate reactions with their respective maximum and minimum value. The typical enzyme loading was set to 5.6-7 μg protein/mg polymer.

FIGS. 4A to 4D show the reaction profile of enzymatic hydrolysis of post-consumer PET with 70% recycled PET content for the enzymes Thf_cut (A), Thc_cut2 (B), Thc_Cut1 (C) and BC-CUT-013 (D). The reactions were carried out for 2 days at 37° C. and pH 7.5 (□), 8 (Δ) and 8.2 (⬆) with 20-25 mg substrate grounded to 0.2-0.5 mm. Hydrolysis products were determined by HPLC. Each value represents the average concentration of total products from duplicate reactions with their respective maximum and minimum value. The typical enzyme loading was adjusted to 5.6-7 μg protein/mg polymer in most of the reactions.

### DETAILED DESCRIPTION OF THE INVENTION

Consequently, the present invention relates in part to a method of degrading recycled polyethylene terephthalate (rPET) comprising the step of subjecting the rPET to at least one cutinase.

The rPET may be provided as single material or as a composite or multilayer material, for example, comprising rPET.

The inventors have obtained, for example, very good results, when the material comprising rPET was a composite PET material comprising 30% or 75% recycled PET.

In accordance with the present invention the rPET is degraded by at least one cutinase. The term “degradation” comprises de-polymerization, which refers to the process of converting a polymer into its final monomers. The term “degradation” more generally describes that the polymer chain is cleaved by at least one of the enzymes, resulting in shorter polymer chains, but not necessary in monomers. This can for example be achieved through the activity of endo-acting enzymes or through the incomplete activity of exo-acting enzymes. In one embodiment of the present invention the method of the present invention may be a method of de-polymerizing rPET, for example at least one rPET-based layer in a packaging.

Cutinases catalyze the reaction of cutine and water to yield cutine monomers. Cutinases are serine esterase, usually containing the Ser, His, Asp triad of serine hydrolases.

The at least one cutinase may be a cutinase from a fungal or microbial source. Using enzymes from a fungal or a microbial source have the advantage that they can be naturally produced, and—in particular, if the enzymes are enzymes that are secreted by the fungus or the micro-organism—the fungus or the micro-organism itself can be used to degrade the at least one polymer layer in a packaging material.

The at least one cutinase may be a cutinase from Thermobifida fusca, Thermobifida cellulosilytica, or Thermobifida alba.

Thermobifida organsims are a thermophilic organism occurring in soil that is a major degrader of plant cell walls in heated organic materials such as compost heaps, rotting hay, manure piles or mushroom growth medium. Its extracellular enzymes have been studied because of their thermostability, broad pH range and high activity.

The inventors have obtained particularly promising results, when the at least one cutinase was selected from the group consisting of Thf_Cut, Thc_Cut1, Thc_Cut2, BC-CUT-013, or combinations thereof.

Thf_Cut (T. fusca), Thc_Cut1 (T. cellulosilytica), Thc_Cut2 (T. cellulosilytica) as well as the 3 metagenomic cutinase BC-CUT-013 were purchased from Biocatalyst Ltd. UK.

The enzymes may be used in pure form. However, the inventors were surprised to see that the enzymes could also be used as crude extracts, for example, as crude extract from a fungal and/or microbial source. Using a crude extract has the advantage, that an expensive purification of the enzymes is not necessary. Consequently, in accordance with the present invention the at least one cutinase may be used as a crude extract. Advantageously, the at least one cutinase may be used as a water soluble, crude extract.

The amount of enzyme used is not critical for the success of the degradation step in the method of the present invention. It is, however, important for the speed of the degradation. The inventors have obtained good results when the degradation was carried out with an enzyme loading of at least about 0.65 μg protein/mg polymer, at least about 6 μg protein/mg polymer, or at least about 50 μg protein/mg polymer.

In particular if the cutinase used in the framework of the present invention is obtainable from a thermophilic organism, the cutinase will also exhibit a certain thermo-stability. Accordingly, the degradation can be carried out at elevated temperatures, for example at a temperature in the range of 30-40° C., 35-45° C. or 40-50° C. The degradation at elevated temperatures will proceed significantly faster. The expected increase in reaction speed can be estimated in accordance with the Arrhenius equation.

However, elevating the reaction temperature will cause costs, for example for the increase in energy usage. Hence, it may be preferred if the degradation is carried out at ambient temperature. This is, in particular, the case if the required reaction time is not critical. Ambient temperature may differ depending, for example, on geographic location and on the season. Ambient temperature may mean for example a temperature in the range of about 0-30° C., for example about 5-25° C.

Accordingly, for example, in the framework of the present invention, the rPET may be subjected to the at least one cutinase at a temperature in the range of 20-50° C., for example 30-40° C. The inventors have obtained very good results at a temperature of about 37° C.

The inventors have further tested the reaction at different pH values. It was found that the method of the present invention was most effective, if the degradation was carried out at neutral to slightly alkaline conditions. Good results were obtained at a pH in the range of 6-9. For example, the rPET may be subjected to the at least one cutinase at a pH in the range of about 6-9, for example in the range of about 6.5-8.

Accordingly, it may be preferred if the degradation is carried out at pH in the range of about 7-9, preferably in the range of about 7.5-8.5, for example at a pH of about 8.2.

The inventors have obtained good results when the rPET was subjected to the at least one cutinase for at least 2 days, for at least 7 days, or for at least 15 days.

With the method of the present invention a partial or even a complete degradation of the rPET appears possible. The inventors conclude this from a corresponding release of monomers and monomer mixtures (TPA, BHET, MHET). For example, it appears possible with the method of the present invention to degrade the rPET by at least 10 weight-%, at least 15 weight-%, at least 20 weight-%, at least 25 weight-%, at least 30 weight-%, at least 35 weight-%, at least 45 weight-%, at least 50 weight-%, or at least 55 weight-%. This degradation resulted in part in the generation of monomers or monomer mixtures. Accordingly, in the method of the present invention the degradation of the at least one polymeric layer results in the generation of at least 10 weight-%, at least 15 weight-%, at least 20 weight-%, at least 25 weight-%, at least 30 weight-%, at least 35 weight-%, at least 45 weight-%, at least 50 weight-%, or at least 55 weight-% of the monomers or monomer mixtures of the degraded polymer.

The method of the present invention is—in particular—well suited for application in packaging recycling. Accordingly, in the framework of the present invention, the rPET may be present in a packaging, for example in rigid or flexible food packaging such as bottles, trays, flexibles or multilayer flexible packing, or pet food packaging such as pouches. For the purpose of the present invention, the term “food” shall be understood in accordance with Codex Alimentarius as any substance, whether processed, semi-processed or raw, which is intended for human consumption, and includes drink, chewing gum and any substance which has been used in the manufacture, preparation or treatment of “food” but does not include cosmetics or tobacco or substances used only as drugs

Multilayer packaging structures are frequently used in the industry today, for example in the food industry. Here, multi-layered packaging is often used to provide light weight packaging with certain barrier properties, strength and storage stability to food items. Such a multi-layered packaging material may be produced by lamination, or coextrusion, for example. Further, techniques based on nanotechnology, UV-treatments and plasma treatments are used to improve the performance of multi-layer packaging. Compr Rev Food Sci Food Saf. 2020; 19:1156-1186 reviews recent advances in multilayer packaging for food applications.

If the packaging comprises a multilayer packaging material, this multilayer packaging material may comprise at least two polymeric layers.

The polymeric layers may comprise a rPET-based layer and at least one layer selected from the group consisting of a further rPET-based layer, a polyurethane (PU)-based layer, a polyethylene (PE)-based layer, or a combination thereof.

A layer shall be considered PU, PE or rPET based, if it contains at least about 50 weight-%, at least about 60 weight-%, at least about 70 weight-%, at least about 80 weight-%, at least about 90 weight-%, at least about 95 weight-%, or at least about 99 weight-% of PU, PE or rPET, respectively.

The polymeric layers may also comprise a rPET layer and at least one layer selected from the group consisting of a further rPET layer, a polyurethane (PU) layer, a polyethylene (PE) layer, or a combination thereof.

PU layers are frequently used in food packaging. PU layers are typically flexible films with high elongation, inherently strong, flexible, and free of plasticizers, that do not become brittle with time. They are resistant to fat and hydrolysis. They can withstand elevated temperatures and exhibit excellent resistance to microbiological attacks.

PET layers are also frequently used in food packaging. They are transparent, have a very good dimensional stability and tensile strength and are stable over wide temperature ranges. PET layers show low water adsorption behavior, are significantly UV-resistant and provide a good gas barrier. Furthermore, it is easy to print on PET in high quality. The moisture barrier properties of PET films are, however, only moderate. For sustainability reasons, rPET is increasingly used to replace virgin PET partially or completely.

Polyethylene (PE) is a plastic polymer that is relatively easy to recycle, nowadays. PE thermoplastics interestingly become liquid at their melting point and do not start to degrade under elevated temperatures. Hence, such thermoplastics can be heated to their melting point, cooled, and reheated again without significant degradation. Upon liquification of PE due to heat, PEs can be extruded or injection molded and—consequently—recycled and used for a new purpose. However, it is problematic to recycle PEs if—e.g., in a multi-layer packaging material—a PE layer is combined with other plastic layers.

One advantage of the method described in the present invention is that it can be used to delaminate selectively rPET layers from a PE layer. Consequently, the method of the present invention may be used for the selective delamination of at least one rPET-based layer in a multilayer packaging.

The inventors could show that the enzyme used in the framework of the present invention could degrade rPET-based layers. For example, the inventors have shown that commercially available rPET containing materials could be degraded with the cutinases used in the framework of the present invention.

In the method of the present invention, the rPET may be present in a packaging comprising a multilayer packaging structure, wherein the multilayer packaging structure comprises a base layer that can be recycled, for example a PE-based layer, and at least one rPET-based layer, wherein the method is used to recycle the multilayer packaging structure by degrading the at least one rPET-based layer and by subjecting the base layer to a recycling stream. The resulting PET monomers can be collected and reused as well.

The inventors further propose that the degradation speed and/or completeness can be significantly increased, if the surface to volume ratio of the packaging, for example the multilayer packaging structure is increased. For example, the packaging may be mechanically treated to reduce the particle size to particles with an average diameter of less than about 5 mm, less than about 1 mm, or less than about 0.5 mm diameter before subjecting the packaging to the enzyme. Typically, the mechanical treatment may be shredding, for example. Hence, the method of the present invention may further comprise the step of reducing the particle size of the rPET and/or the rPET containing material, for example the rPET containing packaging, before or during subjecting the rPET and/or the rPET containing material to at least one cutinase. The particle size may be reduced by a mechanical treatment to particles with an average diameter of less than about 5 mm, less than about 1 mm, or less than about 0.5 mm diameter.

One advantage of the method of the present invention is that it can be carried out under controlled conditions, for example in a closed vessel, such as a bioreactor, for example. The relatively gently conditions of the degradation process do not require bioreactors that can withstand extreme conditions, which in turn contributes to the cost effectiveness of the method of the present invention. Using a closed vessel in turn has the advantage that reaction and process parameters, such as temperature and agitation, for example, can be precisely controlled.

Those skilled in the art will understand that they can freely combine all features of the present invention disclosed herein. In particular, features described for the method of the present invention may be combined. Further, features described for different embodiments of the present invention may be combined.

Although the invention has been described by way of example, it should be appreciated that variations and modifications may be made without departing from the scope of the invention as defined in the claims.

Furthermore, where known equivalents exist to specific features, such equivalents are incorporated as if specifically referred in this specification. Further advantages and features of the present invention are apparent from the figures and non-limiting examples.

### Example 1: Enzymatic Degradation of 30% Recycled PET by Four Cutinases Materials and Chemicals

The polyethylene terephthalate (PET) used for enzymatic assays was post-consumer PET from Henniez still water bottles of 33 cl, with either 30% or 75% recycled PET (rPET). Glycerol, K₂HPO₄, KH₂PO₄, NaOH and ethylacetate, hydrochloric acid, formic acid, hydrochloric acid and methanol were all purchased from Sigma. Terephthalic acid (TPA) was purchased from Fisher Scientific, dimethyl sulfoxide (DMSO) was from Fluka.

Thf_Cut1 (T. fusca), Thc_Cut2 (T. cellulosilytica) and ThcCut1 (T. cellulosilytica) as well as the metagenomic cutinases BC-CUT-013 was purchased from Biocatalyst.

TABLE 1
List of enzymes investigated, their type, abbreviation, organism
of origin, production organism, quality and supplier.

| | | | | Production | | Supplier/ |
|---|---|---|---|---|---|---|
| Name | Abbreviation | Type | Organism | Organism | Quality | Producer |
| T. fusca | Thf_Cut | Cutinase | T. Fusca | Recombinant | Crude | Biocatalysts Ltd. |
| cutinase | | | | E. coli | soluble | UK |
| T. cellulosilytica | Thc_Cut2 | Cutinase | T. cellulosilytica | | extract | |
| cutinase 2 | | | | | | |
| T. cellulosilytica | Thc_Cut1 | Cutinase | T. cellulosilytica | | | |
| 1 | | | | | | |
| Biocatalyst | BC-CUT-013 | Cutinase | Metagenomic | | | |
| Cutinase 013 | | | | | | |

All enzymes were diluted to stock solutions of 1 mg/ml protein in 40% (w/v) glycerol for easier handling during experiments. The final enzyme load corresponded to 5.6-7 μg/mg rPET polymer.

Enzymatic Hydrolysis of Post-Consumer Polyethylene Terephthalate (PET) with Recycled PET Content (rPET)

The post-consumer water bottles with 30% or 75% recycled PET content (rPET) were pre-treated before being submitted to enzymatic treatment. The rPET was cut in squares of 1-2 cm, washed with ethanol (for about 30 min) and dried at 37° C. The rPET was subsequently shredded using a 6870D Freezer/Mill® Cryogenic Grinder from SPEX® SamplePrep. The shredded rPET particles were sieved, separating pieces into four size categories: <0.2 mm, 0.2-0.5 mm, 0.5-1 mm, and >1 mm.

Approximately 20-25 mg of pre-treated post-consumer rPET powder was placed into 2 mL Eppendorf tubes or 4 mL glass vials with a PTFE/silicone/PTFE septum. The reactions were carried out at 37° C. in 1.5 mL freshly prepared enzyme solutions in 100 mM Na₂HPO₄/NaH₂PO₄ buffer at pH 7.

For the 2 mL tubes, the reactions were performed in a ThermoMixer® 5437 from Eppendorf at 1100 rpm, while the glass vials were placed on the horizontal in an ISF1-X incubator shaker from Kuhner Shaker at 100 rpm, to keep the rPET particles in suspension. Control reactions were performed with buffer instead of enzyme solution. Samples for product analysis were taken periodically.

Analysis of rPET Hydrolysis Products by HPLC

The products of the enzymatic hydrolysis of rPET were quantified by high-pressure liquid chromatography (HPLC). Samples of 50 μL were taken periodically from the reaction mixture and transferred to tubes on ice containing 205 μL of 25 mM HCl present in the HPLC mobile phase (0.1% formic acid in 30% MeOH), to stop the reaction and precipitate the enzymes. The samples were then centrifuged at 16,000 g at 0° C., for 15 min. Approximately 200 μL of the supernatant was transferred to HPLC glass vials. The samples were analyzed by reversed phase chromatography using an Agilent 1200 series system, equipped with an Acquity UPLC HSS C18 1.8 μm 2.1×50 mm column from Waters and a diode array detector (DAD), with detection at 241 nm. A volume of 5 or 10 μL sample was injected into the system. The flow was 0.2 mL/min, the column operated at 50° C. and the run time was 8 min. Calibration standards of terephthalic acid (TPA), mono(2-hydroxyethyl terephthalate) (MHET) and bis(2-hydroxyethyl terephthalate) (BHET) were prepared in the same way as samples, with concentrations ranging from 0.005 to 1 mM. Stock solutions of 10 mM of all compounds were prepared in DMSO.

### Results and Discussion

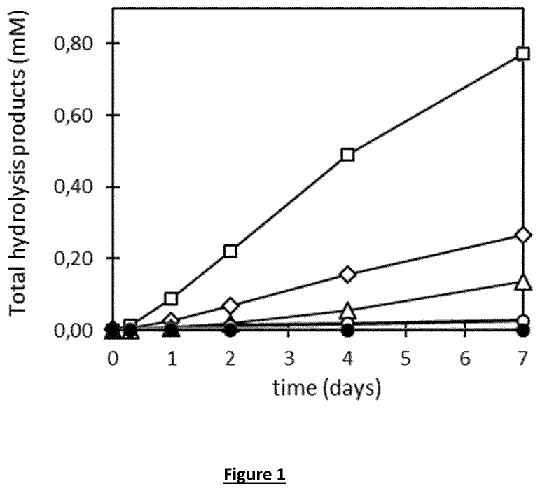

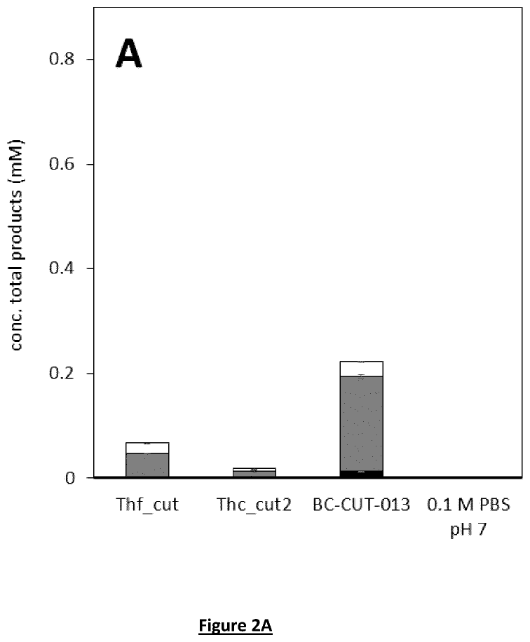

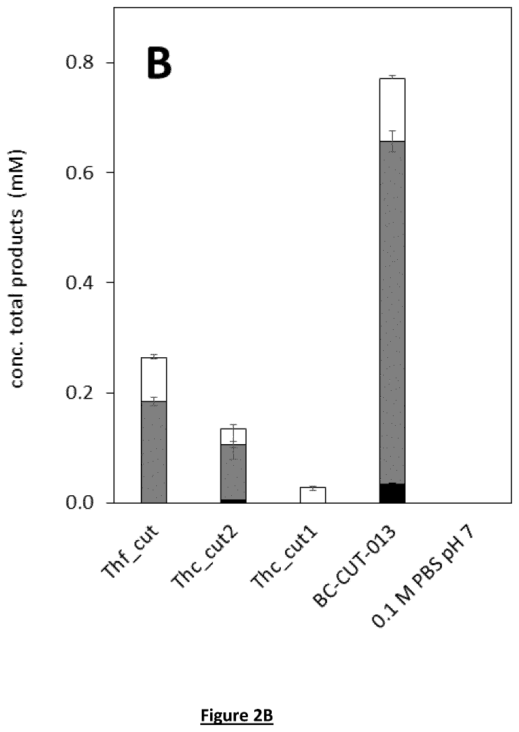

Of all tested enzymes, the highest total product formation for degrading rPET for 7 days at pH 7 and ambient temperatures of 37° C. was detected for BC-CUT-013, a novel cutinase from Biocatalyst (see FIG. 1 and FIG. 2). BC-CUT-013 exceeded the three widely reported PET degrading enzymes Thf_cut, Thc_cut2 and Thc_Cut1 by a factor of three with 0.76 mM after 7 days (see FIG. 1 and FIG. 2b). To the best of the inventor's knowledge, this is the first report on enzymatic hydrolysis of recycled PET (rPET). So far, only the use of post-consumer PET has been reported as substrate for cutinases and enzymes in general (see: Wei, R., et al. 2019, Advanced Science 6(14): 1900491 and Müller, R.-J., et al. 2005, Macromolecular Rapid Communications, 26(17), 1400-1405). The study by Wei et al. 2019 demonstrated an about 4-fold lower efficiency of cutinases on post-consumer PET compared to virgin PET film and the authors linked the behaviour to the difference in crystallinity. However, no information is available about recycled content. In the present invention it was found that the BC-CUT-0013 enzyme from Biocatalysts was not only capable of degrading rPET but also was least affected by the recycling content. As shown in Table 1, up to 21% reduced hydrolysis efficiency was observed when increasing the recycled content from 30% to 75%, while other cutinases (Thc_Cut) showed even higher reduction in hydrolysis of up to 30%. The decrease in hydrolysis efficiency with increasing recycled content of PET is likely to be associated with the higher content of modified PET and crystallinity. Typically, to compensate for the reduced quality of rPET compared to virgin PET that is caused by the reduction in molecular weight through thermal hydrolysis during mechanical recycling, chain extenders are commonly used (D. S. Achilias (Ed.), Mater. Recycl. Trends Perspect., InTech, Rijeka, Croatia (2012), pp. 85-114). Such chemical modification increases the molecular weight and alters the overall physicochemical properties of rPET to regain similar mechanical properties as that virgin PET (see Torres et al. 2001, 79(10), 1816-1824). Also, the higher the recycled content the higher the cross-linked PET chains, which may affect the accessibility of the enzyme (cutinase) to the chains. Notably, reactions in literature were performed at much higher temperatures, while the present invention conducted reactions at ambient temperatures, which further underlines the uniqueness and importance of the results as it not only offers solutions to recycle rPET in multilayers but also to do so at lower temperatures that would bring about energy savings compared to reactions described by the prior art.

Table 1 shows the effect of recycled PET with content of 75% in post-consumer PET packaging on BC-CUT-013 and Thf_Cut hydrolysis efficiency. The reactions were carried out in glass vials at 37° C. and pH 7 for 24 h using 20-25 mg grounded rPET to 0.2-0.5 mm. The typical enzyme loading was set to 7 μg protein/mg polymer. Hydrolysis products were quantified by HPLC.

| | Reduction in hydrolysis rate between |
|---|---|
| Enzyme/Cutinase | 30% and 75% rPET after 24 h |
| BC-CUT-013 | 21 ± 2% |
| Thf_Cut | 30 ± 4% |

### Example 2: Impact of pH on Enzymatic Degradation Reactions on 75% Recycled

PET

### Material and Methods

### Materials and Chemicals

The polyethylene terephthalate (PET) used for enzymatic assays was post-consumer PET from Henniez still water bottles of 33 cl, with either 30% recycled PET (rPET). Glycerol, K₂HPO₄, KH₂PO₄, NaOH and ethylacetate, formic acid, hydrochloric acid and methanol were all purchased from Sigma. Terephthalic acid (TPA) was purchased from Fisher Scientific, dimethyl sulfoxide (DMSO) was from Fluka.

Thf_Cut1 (T. fusca), Thc_Cut2 (T. cellulosilytica), Est119 (T. alba) and ThcCut1 (T. cellulosilytica) as well as the metagenomic cutinases BC-CUT-013 was purchased from Biocatalyst (see Table 2).

TABLE 2
List of enzymes investigated, their type, abbreviation, organism
of origin, production organism, quality and supplier.

| | | | | Production | | Supplier/ |
|---|---|---|---|---|---|---|
| Name | Abbreviation | Type | Organism | Organism | Quality | Producer |
| T. fusca | Thf_Cut | Cutinase | T. Fusca | Recombinant | Crude | Biocatalysts Ltd. |
| cutinase | | | | E. coli | soluble | UK |
| T. cellulosilytica | Thc_Cut2 | Cutinase | T. cellulosilytica | | extract | |
| cutinase 2 | | | | | | |
| T. cellulosilytica | Thc_Cut1 | Cutinase | T. cellulosilytica | | | |
| 1 | | | | | | |
| Biocatalyst | BC-CUT-013 | Cutinase | Metagenomic | | | |
| Cutinase 013 | | | | | | |

All enzymes were diluted to stock solutions of 1 mg/ml protein in 40% (w/v) glycerol for easier handling during experiments except for Pseudomonas cepacia Lipase which was diluted to 0.1 mg protein/ml due to higher purity.

### Enzymatic Hydrolysis of Post-Consumer Polyethylene Terephthalate (PET)

The post-consumer PET bottles with 75% recycled content were pre-treated before being submitted to enzymatic treatment. The PET was cut in squares of 1-2 cm, washed with ethanol (for about 30 min) and dried at 37° C. The PET was subsequently shredded using a 6870D Freezer/Mill® Cryogenic Grinder from SPEX® SamplePrep. The shredded PET was sieved, separating pieces into four size categories: <0.2 mm, 0.2-0.5 mm, 0.5-1 mm, and >1 mm.

Approximately 20-25 mg of pre-treated post-consumer rPET powder was placed into 2 mL Eppendorf tubes or 4 mL glass vials with a PTFE/silicone/PTFE septum. The reactions were carried out at 37° C. in 1.5 mL freshly prepared enzyme solutions in 100 mM Na₂HPO₄/NaH₂PO₄ buffer at pH 7.5, 8, 8.2. The final enzyme load corresponded to 5.6-7 μg/mg polymer.

For the 2 mL tubes, the reactions were performed in a ThermoMixer® 5437 from Eppendorf at 1100 rpm, while the glass vials were placed on the horizontal in an ISF1-X incubator shaker from Kuhner Shaker at 100 rpm, to keep the PET particles in suspension. Control reactions were performed with buffer instead of enzyme solution. Samples were taken after every 24 h.

At the end of the reactions, the PET was washed two times with MilliQ and one time with ethanol dried at room temperature and stored for further analysis using size exclusion chromatography (SEC).

### Analysis of Hydrolysis Products by HPLC

The products of the enzymatic hydrolysis of rPET were quantified by high-pressure liquid chromatography (HPLC). Samples of 50 μL were taken and transferred to tubes on ice containing 205 μL of 25 mM HCl in the HPLC mobile phase (0.1% formic acid in 30% MeOH), to stop the reaction and precipitate the enzymes. The samples were then centrifuged at 16000 g at 0° C., for 15 min. Approximately 200 μL of the supernatant was transferred to HPLC glass vials. The samples were analyzed by reversed phase chromatography using an Agilent 1200 series system, equipped with an Acquity UPLC HSS C18 1.8 μm 2.1×50 mm column from Waters and a diode array detector (DAD), with detection at 241 nm. A volume of 5 or 10 μL sample was injected into the system. The flow was 0.2 mL/min, the column operated at 50° C. and the run time was 8 min. Calibration standards of terephthalic acid (TPA), mono(2-hydroxyethyl terephthalate) (MHET) and bis(2-hydroxyethyl terephthalate) (BHET) were prepared in the same way as samples, with concentrations ranging from 0.005 to 1 mM. Stock solutions of 10 mM of all compounds were prepared in DMSO.

### Results and Discussion

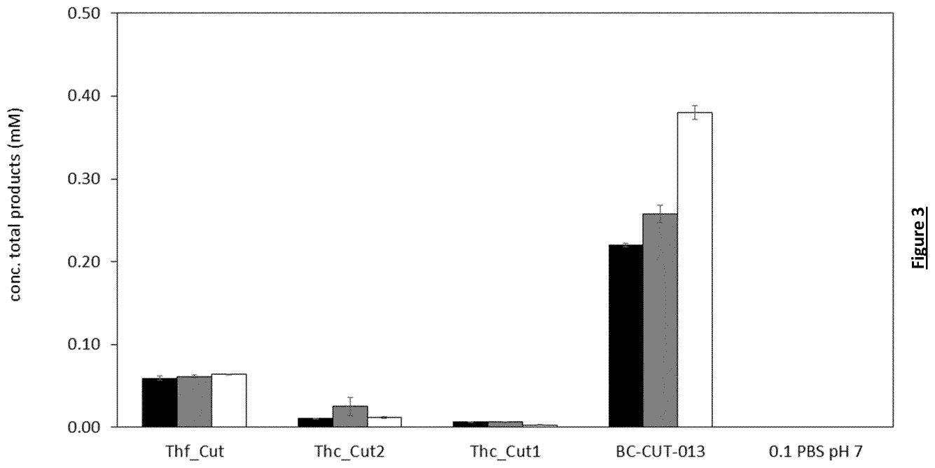

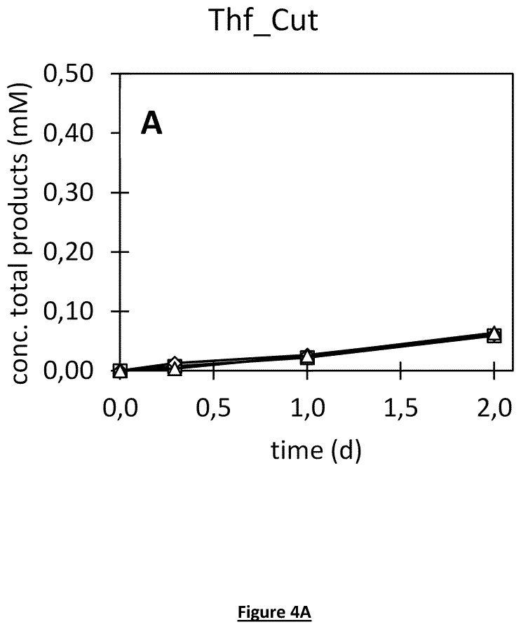

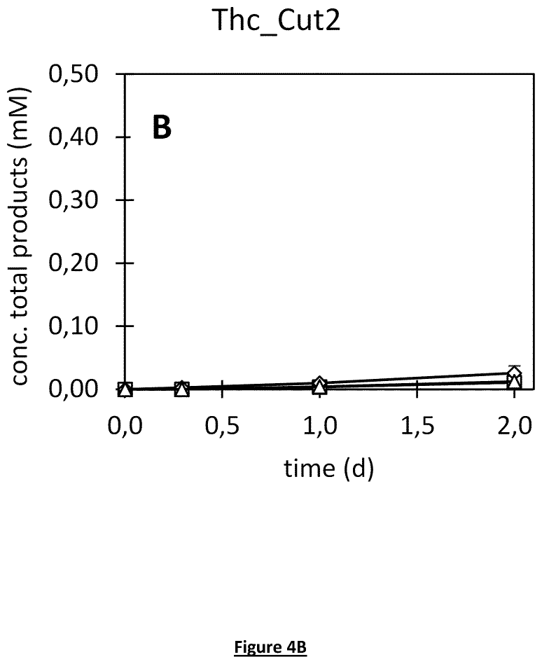

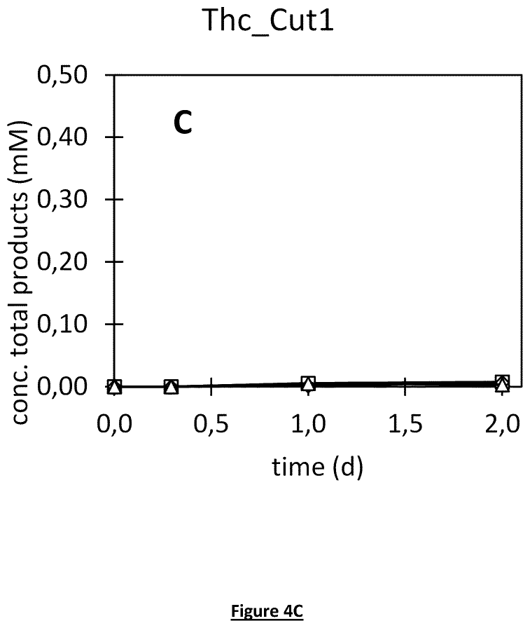

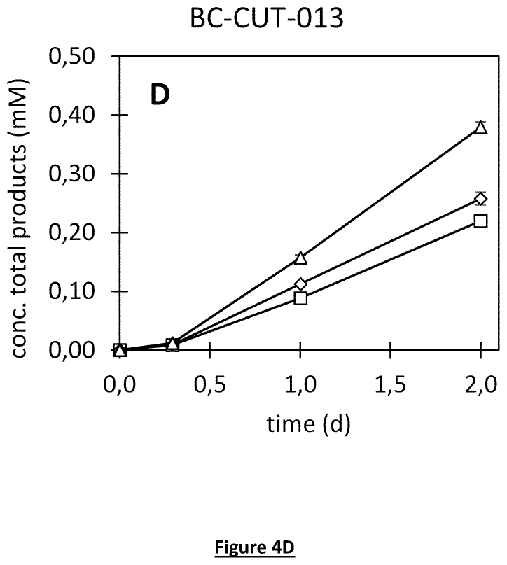

Assessing the pH dependency of the different cutinases for the activity towards 75% rPET, the inventors found that the activity of BC-CUT-013 can be increased by almost factor 2 when changing the reaction pH from 7.5 to 8.2 (see FIG. 3). Note, the control reaction without enzyme did not show hydrolysis products (FIG. 3). The other cutinases showed hardly any change in hydrolysis rate when varying the reaction pH (see FIG. 3). Thus, despite having a broad pH range (see Table 3), these cutinases (Thf-Cut, Thc_Cut2, Thc_Cu1) feature an overall much lower hydrolysis activity on rPET compared to BC-CUT-013. This means increasing the pH to 8.2 can significantly improve the hydrolysis reaction of PET by this particular enzyme (BC-CUT-013) and thus serves as an important parameter for the optimization of rPET hydrolysis.

Table 3 below summarizes the pH the optimal ranges of enzymatic degradation of post-consumer 75% recycled PET for four cutinases (Thf_Cut, Thf_Cut2, Thc_Cut1 and BC-CUT013). The reaction pH was set to 7.5, 8 and 8.2, respectively, for 48 h in 4 ml glass vials at 37° C. with 20-25 mg rPET grounded to 0.2-0.5 mm.

| Enzyme | pH -optimum |
|---|---|
| Thf_Cut | pH 7.5-8.2 |
| Thc_Cut2 | pH 7-8.2 |
| Thc-Cut1 | — |
| BC-CUT-13 | pH 8.2 |

1. Method of degrading recycled polyethylene terephthalate (rPET) comprising the step of subjecting the rPET to at least one cutinase.

2. Method in accordance with claim 1, wherein the at least one cutinase is selected from the group consisting of Thf_Cut, Thc_Cut1, Thc_Cut2, BC-CUT-013, or combinations thereof.

3. Method in accordance with claim 1 wherein the at least one cutinase is used as a crude extract.

4. Method in accordance with claim 1 wherein the at least one cutinase is used with an enzyme loading of at least 0.65 μg protein per mg polymer.

5. Method in accordance with claim 1 wherein the rPET is subjected to the at least one cutinase at a temperature in the range of 20-50° C.

6. Method in accordance with claim 1 wherein the rPET is subjected to the at least one cutinase at a pH in the range of about 6-9.

7. Method in accordance with claim 1 wherein the rPET is subjected to the at least one cutinase for at least 2 days.

8. Method in accordance with claim 1, wherein the rPET is present in a packaging.

9. Method in accordance with claim 8, wherein the packaging comprises a multilayer packaging structure comprising at least two polymeric layers, wherein the polymeric layers comprises a rPET-based layer and at least one layer selected from the group consisting of a polyurethane (PU)-based layer, a polyethylene terephthalate (PET)-based layer, a polyethylene (PE)-based layer, or a combination thereof.

10. Method in accordance with claim 1, wherein the method is used for the selective delamination of at least one rPET-based layer in a multilayer packaging.

11. Method in accordance with claim 1, wherein the rPET is present in a packaging comprising a multilayer packaging structure, wherein the multilayer packaging structure comprises a base layer that can be recycled, and at least one rPET layer, wherein the method is used to recycle the multilayer packaging materials by degrading the at least one rPET-based layer and by subjecting the base layer to a recycling stream.

12. Method in accordance with claim 1, wherein the method further comprises the step of reducing the particle size of the rPET and/or the rPET containing material, before or during subjecting the rPET and/or the rPET containing material to at least one cutinase.

13. Method in accordance with claim 12, wherein the particle size is reduced by a mechanical treatment to particles with an average diameter of less than about 5 mm.

14. Method in accordance with claim 1, wherein the method is carried out in a closed vessel.
